(19) United States
(12) Patent Application Publication
(10) Pub. No.: US 2003/0031724 A1
(43) Pub. Date: Feb. 13, 2003

(54) Anti-inflammatory composition and method of using same

(76) Inventors: Frank Orthoefer, Cordova, TN (US); Paul Binford, Chapmansboro, TN (US); Robert Nicolosi, Lowell, MA (US)

Correspondence Address: Robert M. Schulman, Esq., Hunton & Williams, 1900 K Street, N.W., Suite 1200, Washington, DC, 20006, US

(21) Appl. No.: 09/855,725

(22) Filed: May 16, 2001

(51) Int. Cl. A61K 35/12

(52) U.S. Cl. 424/522

(57) ABSTRACT

Compositions that may be cost-efficiently derived or processed from an emu and used for reducing and/or preventing inflammation in patients are disclosed. The compositions comprise colorless emu oil and/or active components of same in certain proportions. Methods of using said compositions to reduce and/or prevent inflammation in patients and methods of preparing said compositions are also disclosed.

### FIELD OF THE INVENTION

The present invention relates generally to compositions that may be cost-effectively derived or processed from the emu, Dromiceius novaehollandiae, and used as anti-inflammatory agents in patients. In particular, the present invention relates to compositions comprising colorless emu oil and/or the active component(s) of same, as well as methods of using said compositions as anti-inflammatories and methods of preparing said compositions.

### BACKGROUND OF THE INVENTION

Emu oil is derived from Dromiceius novaehollandiae, commonly referred to as the emu, a large flightless bird native to Australia and resembling an ostrich. The emu is a member of the Ratite family of birds and is the only member of that family to have a subcutaneous layer of fat found just under the skin on the back of the bird. Emus have been a commercially valuable bird for a number of years. For example, the meat of an emu is red meat, similar to beef from cattle, only lower in cholesterol and calories, and higher in protein. The skin of the emu can be used as a very high quality, durable leather. The feathers of the emu are useful in ornamental items, such as jewelry, and in fishing lures.

The oil from the emu has only recently been recognized as having potential commercial value. Various compositions containing emu oil and a variety of uses for emu oil have been disclosed. For example, Fankhauser et al., U.S. Pat. No. 6,162,447, disclose a microstructured cosmetic composition comprising: A) a cosmetically acceptable oil phase which is dispersed in; B) an aqueous phase comprising 0.05 to 3.0, preferably 0.2 to 1.0% by weight, based on the weight of the total composition, of a .beta.-1,3-glucan having a mean molecular weight within the range of from 1.times.10.sup.6 to 12.times.10.sup.6, preferably from 2.times.10.sup.6 to 10.times.10.sup.6; the oil phase A) in the microstructured cosmetic composition having a mean particle size within the range of from 0.05 to 1000 microns.

Hart, U.S. Pat. No. 6,133,318, discloses single medicine oxalic acid or oxalate composition or “magic bullet” and method of treatment or prevention of warm-blooded animals including humans and pets for infectious or pathogenic microbial, bacterial, or viral disease, chemoprevention of bacterial or viral infections, and the like, is provided which includes at least one therapeutically effective form of oxalic acid or oxalate selected from oxalic acid in a free acid, ester, lactone or salt form and oxalate including sodium oxalate, oxalic acid dihydrate, anhydrous oxalic acid, oxamide, and oxalate salts, natural or processed foods including molds, plants or vegetables containing oxalic acid or oxalate, beverages, liquids or juices containing oxalic acid or oxalate, additives containing oxalic acid or oxalate, and combinations thereof. The composition may also contain a pharmaceutically acceptable carrier or diluent for the therapeutically effective form of oxalic acid or oxalate. Methods are provided including the steps of periodically administering, by topical, oral, or parenteral application, a therapeutically effective dosage of a composition including at least one therapeutically effective form of oxalic acid or oxalate and improving chemotherapy reducing the intake of oxalic acid or oxalate blockers such as citric acid, ascorbic acid, (vitamin C), pyridoxine hydrochloride (vitamin B6), calcium, alcohol, resins, clays, foods containing calcium, beverages containing alcohol, citric acid, or ascorbic acid, red meat or white meat of fowl containing pyridoxine hydrochloride, or other foods nutritional supplements or beverages containing oxalic acid or oxalate blockers.

Kang et al., U.S. Pat. No. 6,106,815, disclose a shampoo composition containing from 0.05% to 10% by weight of capsules or particles of 0.2 to 6.0 mm in diameter which include oily substances. The disclosed composition also includes from 5% to 65% by weight of surfactants, from 0.15% to 2% by weight of copolymer of methyl vinyl ether/maleic anhydride decadiene crosspolymer and from 0.1% to 2.0% by weight of polymeric conditioners. The reference discloses that the presence of the crosspolymer significantly enhances the dispersion and stability properties of the capsules.

Tisdale et al., U.S. Pat. No. 6,103,246, disclose a composition which is a cream or lotion for topical application to human skin. The composition includes emu oil as a component. Emu oil is generally comprised of myristic acid, palmitic acid, palmitoleic acid, margaric acid, stearic acid, elaidic acid, oleic acid, vaccenic acid, linoleic acid, linolenic acid, arachidic acid and eicosenoic acid. The composition also includes a medically active component, such as lactic acid or some other alpha-hydroxy acid. The balance of the components of the composition are water, glycerin, and an emulsion base. Liposomes may be included in the composition. The composition provides for increased skin penetrability due to the emu oil.

Macrides et al., U.S. Pat. No. 6,083,536, discloses an anti-inflammatory, and particularly anti-arthritic, treatment of a human or animal patient comprises administration to the patient of an effective amount of a lipid extract of Perna canaliculus or Mytilus edulis.

Cardinale Fezler, U.S. Pat. No. 5,989,594, discloses an adenosine triphosphate (ATP) deficiency as the cause of many autoimmune, muscle and bone wasting disorders in humans and animals. Ratite muscle and bone extracts provide a rich source of ATP and regulatory proteins which can be used in treating conditions associated with ATP deficiency.

Holick, U.S. Pat. No. 5,958,384, discloses that topical or parenteral administration of emu oil to a mammal stimulates the proliferation of skin. Emu oil can be used to treat skin wrinkles and rejuvenate aged and photo-damaged skin. It has also been discovered that emu oil can be topically applied to stimulate melanogenesis in the skin and to stimulate hair growth. Thus, emu oil is useful to treat pigmentation disorders such as hypopigmentation, stimulating melanogenesis to enhance skin tanning, and treating disorders relating to disturbances in hair cycling such as alopecia, male pattern baldness, female baldness, and chemotherapy-induced alopecia.

Manker et al., U.S. Pat. No. 5,929,113, disclose a method for repelling biting insects such as mosquitoes, ticks, and biting flies by topically applying to the skin of a subject emu oil, fractions of emu oil obtained by flash chromatography or a composition comprising emu oil or active fractions thereof and at least one other insect repellent such as citronella, DEET or saponin. Also provided are compositions for repelling biting insects comprising diluted fractions of emu oil, and compositions comprising emu oil or active fractions thereof and at least one other insect repellent such as citronella, DEET or saponin.

Rivlin, U.S. Pat. No. 5,849,334, discloses compositions comprising a local anesthetic and emu oil. The compositions are useful in a method of anesthetizing cornified skin comprising topically administering the subject compositions. The compositions and methods permit rapid and effective anesthesia of cornified skin, such that various surgeries and interventions can be performed without the need for subcutaneous injections of local anesthetics. Further disclosed is a compartmentalized kit having a first container providing emu oil and a second container providing an aqueous solution of a local anesthetic.

Manker et al., U.S. Pat. No. 5,801,196, disclose a method for repelling biting insects such as mosquitoes by topically applying to the skin of a subject fractions of emu oil obtained by flash chromatography. Also disclosed are methods for repelling biting insects by topically applying diluted fractions of emu oil. Further disclosed is a compound useful in repelling biting insects having the NMR spectrum of FIG. 3, that is ultra violet light sensitive and is reactive to vanillin/sulfuric acid, and compounds useful in repelling biting insects having the NMR spectrum of FIG. 4, that are not ultra violet light sensitive and are not reactive to vanillin/sulfuric acid.

Kousoulas et al., U.S. Pat. No. 5,786,179, discloses a method of enhancing the production performance of avians, by administering to a bird a heterologous protein comprised of inhibin protein, or a fragment thereof, and a carrier protein. Further disclosed is a method of enhancing the production performance of avians, by administering to a bird a fusion gene product comprising a gene encoded for the expression of alpha-subunit avian inhibin protein, or a fragment thereof, and a gene encoded for the expression of a carrier protein. An effective amount of the heterologous protein or fusion gene product is administered to an animal such that an immunological response occurs in the animal against the heterologous protein. Also disclosed is the above heterologous protein and fusion gene product, and methods of producing the same.

Holick, U.S. Pat. No. 5,744,128, discloses that topical or parenteral administration of emu oil to a mammal stimulates the proliferation of skin. Emu oil can be used to treat skin wrinkles and rejuvenate aged and photo-damaged skin. It has also been discovered that emu oil can be topically applied to stimulate melanogenesis in the skin and to stimulate hair growth. Thus, emu oil is useful to treat pigmentation disorders such as hypopigmentation, stimulating melanogenesis to enhance skin tanning, and treating disorders relating to disturbances in hair cycling such as alopecia, male pattern baldness, female baldness, and chemotherapy-induced alopecia.

Rivlin, U.S. Pat. No. 5,698,227, discloses compositions comprising lidocaine and emu oil. The compositions are disclosed as being useful in a method of anesthetizing cornified skin comprising topically administering the subject compositions. The compositions and methods permit rapid and effective anesthesia of cornified skin, such that various surgeries and interventions can be performed without the need for subcutaneous injections of local anesthetics. Further disclosed is a compartmentalized kit having a first container providing emu oil and a second container providing an aqueous solution of lidocaine.

Thacker, U.S. Pat. No. 5,688,746, discloses lubricant and grease compositions comprising ratite oils. These lubricant and grease compositions include compositions comprising a major amount of an oil of lubricating viscosity, and a minor amount of ratite oil. The lubricating and grease compositions are disclosed as being useful as lubricants, friction reducers, and coolants.

Manker et al., U.S. Pat. No. 5,677,338, disclose a method for repelling biting insects such as mosquitoes by topically applying to the skin of a subject fractions of emu oil obtained by flash chromatography. Also disclosed are methods for repelling biting insects by topically applying diluted fractions of emu oil. The reference also discloses a compound useful in repelling biting insects having the NMR spectrum of FIG. 3, that is ultra violet light sensitive and is reactive to vanillin/sulfuric acid. Also disclosed are compounds useful in repelling biting insects having the NMR spectrum of FIG. 4, that are not ultra violet light sensitive and are not reactive to vanillin/sulfuric acid.

Fein et al., U.S. Pat. No. 5,662,921, disclose emu oil being therapeutically used in methods for lowering cholesterol, triglycerides and low density lipoproteins and increasing high density lipoproteins; preventing and treating allergies; preventing scarring; treating headaches; preventing nose bleeds; treating and preventing cold and flu symptoms; and relieving discomfort associated with menstruation. Additionally, emu oil is disclosed as acting as an effective chemical buffer in combination with glycolic acid.

Marrone et al., U.S. Pat. No. 5,626,882, disclose a method for repelling biting insects such as mosquitoes by topically applying emu oil to the skin.

Fein et al., U.S. Pat. No. 5,472,713, disclose emu oil as being therapeutically used in methods for lowering cholesterol, triglycerides and low density lipoproteins and increasing high density lipoproteins; preventing and treating allergies; preventing scarring; treating headaches; preventing nose bleeds; treating and preventing cold and flu symptoms; and relieving discomfort associated with menstruation. Additionally, emu oil is disclosed as acting as an effective chemical buffer in combination with glycolic acid.

Ghosh et al., U.S. Pat. No. 5,431,924, discloses a biologically active component of emu oil that is useful in pharmaceutical composition for the treatment of inflammation of environmental and systemic origins. Pharmaceutical compositions including emu oil and dermal transport enhancing compounds are also disclosed as useful topical anti-inflammatory treatments.

However, none of the above-discussed references discloses effective and cost-efficient anti-inflammatory compositions and/or methods. In view of the deficiencies of the various products and processes disclosed in the above-discussed references, it is highly desirable to provide compositions that are cost-efficient and effective as anti-inflammatories. Further, it is highly desirable to provide compositions derived and/or refined from emu oil for use as anti-inflammatories, as well as methods for using said compositions and methods for preparing said compositions. It is also highly desirable to provide such compositions in a cost-efficient manner.

### SUMMARY OF THE INVENTION

The present invention provides effective and cost-efficient anti-inflammatory compositions, as well as methods for preparing and/or using same. The present invention further provides compositions derived from emu oil, as well as methods for preparing and/or using same.

An embodiment of the present invention provides a composition for reducing and/or preventing inflammation in a patient comprising: colorless emu oil, wherein the colorless emu oil reduces and/or prevents inflammation in the patient when administered in an effective amount to said patient.

A further embodiment of the present invention provides a composition prepared by a process comprising: obtaining subcutaneous fat from an emu; and processing the subcutaneous fat at a low temperature in the absence of oxygen to obtain colorless emu oil.

An even further embodiment of the present invention provides a method for reducing and/or preventing inflammation in a patient comprising: administering to the patient an effective amount of a composition comprising colorless emu oil.

A still further embodiment of the present invention provides a method for preparing a composition for reducing and/or preventing inflammation in a patient comprising: obtaining subcutaneous fat from an emu; and processing the subcutaneous fat at a low temperature in the absence of oxygen to obtain colorless emu oil.

### BRIEF DESCRIPTION OF THE DRAWINGS

FIG. 1 is a graph of comparative average and standard deviation data from the study of the anti-inflammatory properties of refined emu oil as observed in mice.

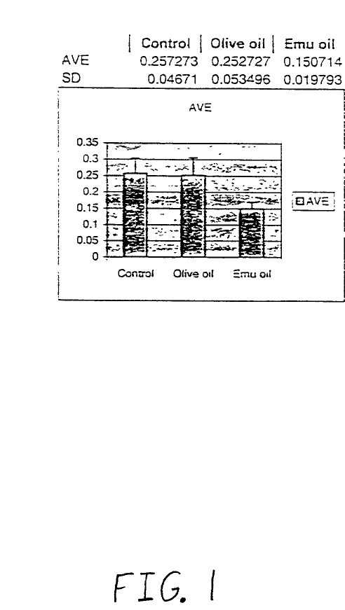

FIG. 2 is a graph comparing the effect of refined emu oil to the effect of other oils and fats on inflammatory induced auricular thickness in CD-1 mice.

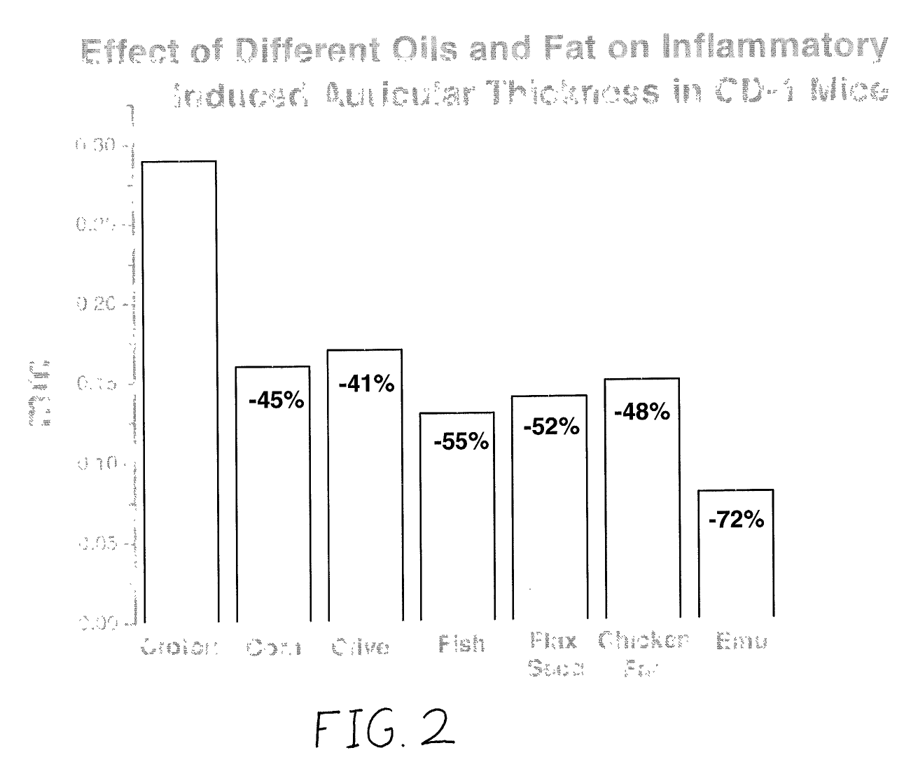

FIG. 3 is a graph comparing the effect of refined emu oil to the effect of other dietary oils and fats on inflammatory induced increases in auricular weight in CD-1 mice.

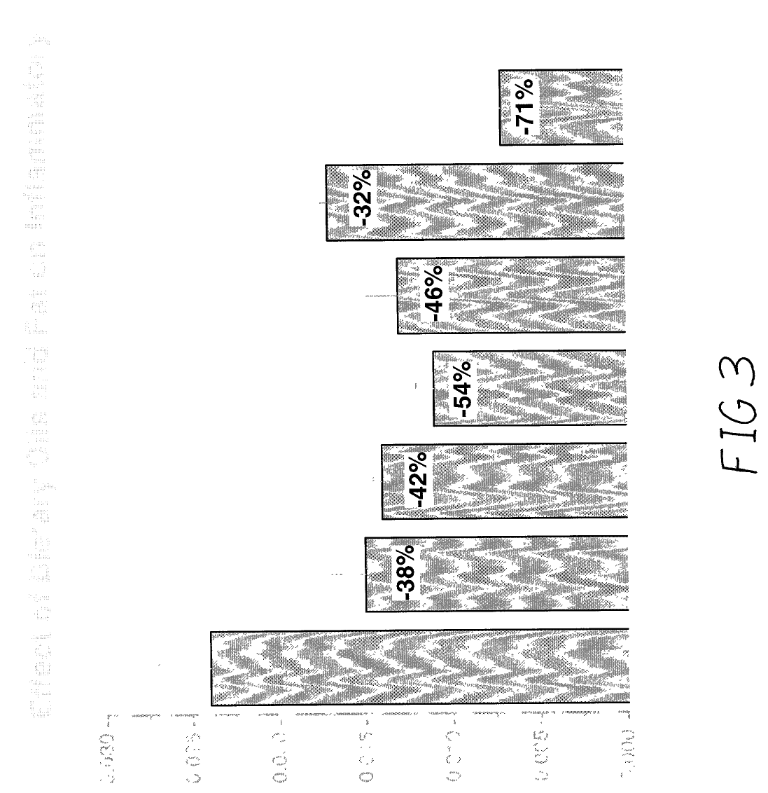

FIG. 4 is a graph comparing the effect of refined emu oil to the effect of other dietary oils on auricular levels of the cytokine TNF-a in CD-1 mice.

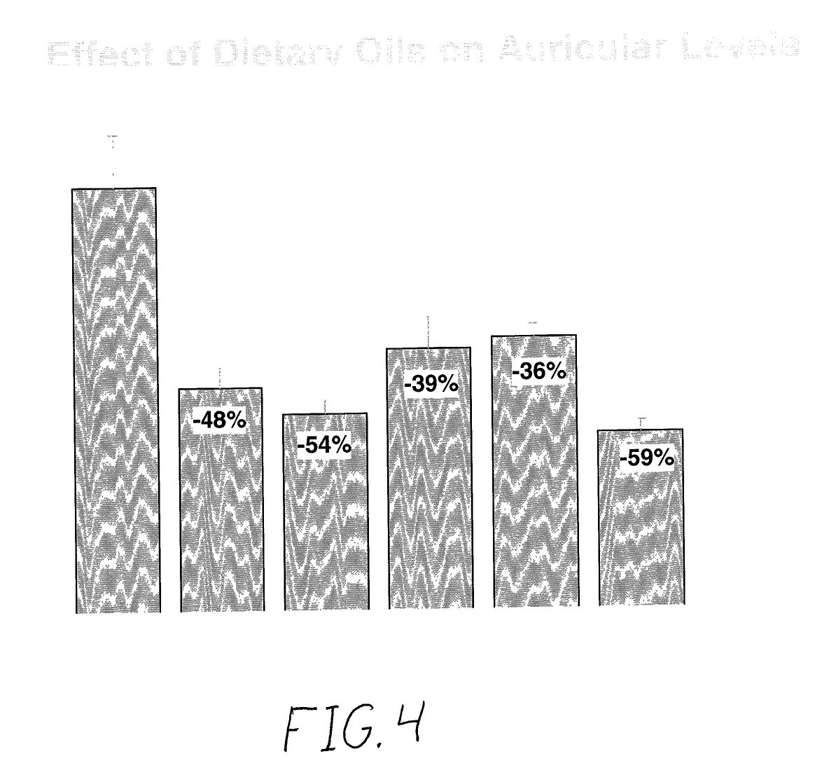

FIG. 5 is a graph comparing the effect of refined emu oil to the effect of other dietary oils on auricular level of IL-1a in CD-1 mice.

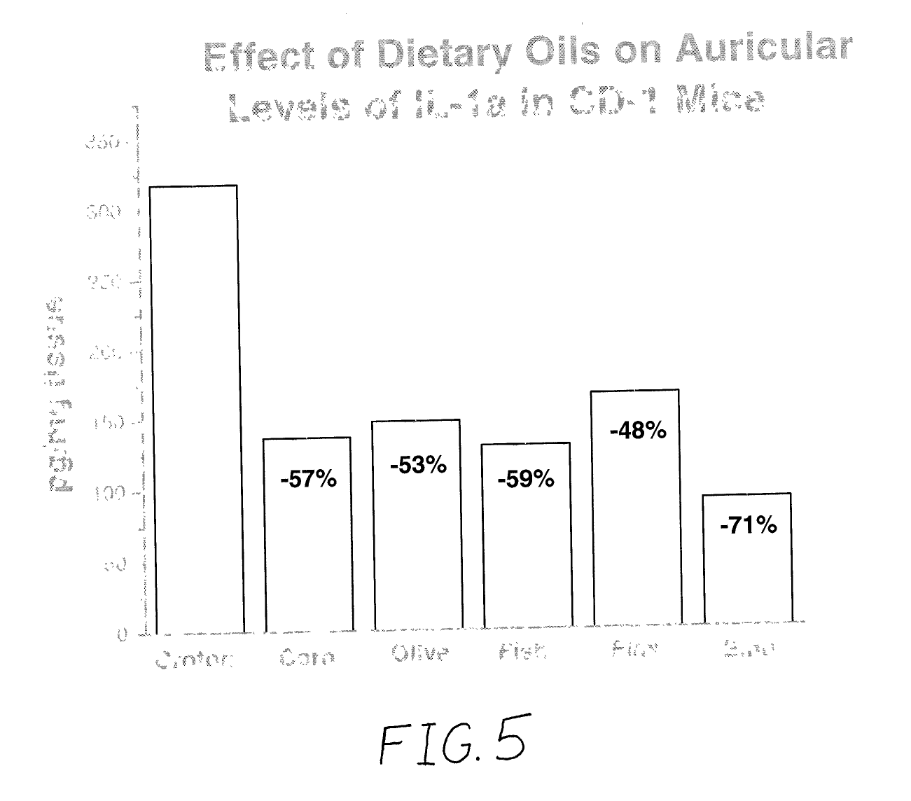

### DETAILED DESCRIPTION OF THE PREFERRED EMBODIMENTS

As used herein the term “therapeutic composition” means a composition useful for treating cells, tissues, organs or systems, both internally and externally.

As used herein the term “therapeutically effective amount” means an amount effective to treat the target medical condition.

The term “pharmaceutically-acceptable”, as used herein, means that the compositions or components thereof so described are of sufficiently high purity and suitable for use in contact with skin, tissues, or membranes without undue toxicity, incompatibility, instability, allergic response, and the like.

It has unexpectedly been found that certain compositions, and particularly compositions refined from crude emu oil and/or subcutaneous fat from the emu, are highly effective as anti-inflammatories. The compositions comprise colorless emu oil or emu oil having substantially no pigment and/or the active components of said colorless emu oil.

Preferably, the colorless emu oil has an American Oil Chemist's Society (AOCS) color rating of Cc13b-45 using the Lobibond method. Compositions from any source, including synthetic compositions, having the herein recited characteristics and properties are contemplated by the present invention. Preferably, the source of the compositions is emu oil.

It is believed that during an inflammatory process, depending on the severity and the cause, the defense mechanisms of the body respond in multiple ways. The cardinal signs of inflammation are: rubor (redness), calor (increased heat), tumor (swelling), dolor (pain) and functio laesa (loss of function). Different signs manifest in different degrees and ways. Cell biology studies reveal that this complex reaction is brought forward by a group of pro-inflammatory cytokines. Inflammation is the body's reaction to invasion by an infectious agent, antigen challenge or even physical damage. This inflammatory process is actively orchestrated by the cytokines, IL-1α, TNFα and IL-6. The mechanism of action by which emu oil reduces inflammation, however, is not well understood.

Emu oil is obtained from Dromiceius novaehollandiae, commonly referred to as the emu. Although the emu is native to Australia, the use of emus from any region of the world is contemplated by the present invention provided the recited characteristics and properties of the emu oil, as disclosed herein, is achieved. Regional variations in the raising, feeding and breeding of emus can affect the characteristics and properties of the emu oil. Preferably, the emus from which the emu oil is extracted are raised and bred in North America on a traditional North American emu diet. More preferably, the emus from which the emu oil is extracted are raised and bred in the 48 contiguous states of the United States. Even more preferably, the emus from which the emu oil is extracted are raised and bred in the Southern and Midwestern United States.

The emu has a subcutaneous layer of fat extending along the back of the bird. The emu may be processed using conventional processing means to extract the subcutaneous layer of fat. An example of the subcutaneous layer of fat and/or crude emu oil is then processed to obtain colorless emu oil. The colorless emu oil may be further refined in a variety of ways.

Preferably, the composition of the present invention comprises about 10% to about 30% by weight of unsaturated essential fatty acids having 16 carbon atoms, about 40% to about 60% by weight of saturated essential fatty acids having 18 carbon atoms and one double bond, and about 10% to about 20% by weight of saturated essential fatty acids having 18 carbon atoms and two double bonds. More preferably, the composition of the present invention comprises about 15% to about 25% by weight of unsaturated essential fatty acids having 16 carbon atoms, about 45% to about 55% by weight of saturated essential fatty acids having 18 carbon atoms and one double bond, and about 14% to about 18% by weight of saturated essential fatty acids having 18 carbon atoms and two double bonds. Even more preferably, the composition of the present invention comprises about 20% by weight of unsaturated essential fatty acids having 16 carbon atoms, about 50% by weight of saturated essential fatty acids having 18 carbon atoms and one double bond, and about 16% by weight of saturated essential fatty acids having 18 carbon atoms and two double bonds.

In accordance with an implementation of the present invention, the compositions of the present invention comprise: oleic acid and linoleic acid; wherein the ratio of the oleic acid to the linoleic acid ranges from about 2:1 to about 4:1. Preferably, the ratio of the oleic acid to the linoleic acid is about 3:1.

In accordance with another implementation of the present invention, the compositions of the present invention comprise: palmitic and/or palmitoleic acid; oleic acid; linoleic acid; and wherein the ratio of the palmitic and/or palmitoleic acid to the oleic acid ranges from about 1:2 to about 1:3. Preferably, the ratio of the palmitic and/or palmitoleic acid to the oleic acid is about 1:2.5.

In a further implementation of the present invention, a method for preparing a composition for reducing and/or preventing inflammation in a patient comprises: obtaining subcutaneous fat from an emu; processing the subcutaneous fat at a low temperature with the absence of oxygen to obtain colorless emu oil.

The subcutaneous fat is obtained from the emu using conventional methods. Preferably, the emu fat is harvested from the emu at the processing facility in such a manner as to prevent contamination of the emu fat. The emus are preferably bled with carcass elevated to ensure as little blood as possible is exposed to the hides and fat area. After removal of hide, fat is cut/trimmed off the carcass with care taken to avoid contamination with blood or other foreign matter. Internal fat is again removed without contaminates of other foreign matters. The fat is then vacuum packed and stored in freezers at low temperature to ensure that it remains as fresh as possible.

The processing of the subcutaneous fat into emu oil is also performed using conventional methods for processing subcutaneous fat into emu oil. It is critical, however, that the reaction conditions under which the processing occurs include a low temperature and an absence of oxygen. Under other reaction conditions (e.g., high temperature and/or in the presence of oxygen), the resulting emu oil is a yellow to gold color and does not have the properties and/or characteristics of the colorless emu oil of the present invention. Preferably, the processing of the subcutaneous fat occurs at a temperature of about 150 degrees F. to about 200 degrees F. More preferably, the processing occurs at a temperature of about 150 degrees F. to about 180 degrees F. The emu oil produced in this manner is colorless emu oil. Preferably, the colorless emu oil has an American Oil Chemist's Society (AOCS) color rating of Cc13b-45 using the Lobibond method.

A variety of conventional methods, including physical refining and chemical refining are used to process the emu oil and/or subcutaneous fat into the compositions in accordance with the present invention. Preferably, physical refining is used in accordance with the present invention. Persons of skill in the art would readily be able to prepare compositions in accordance with the present invention, based upon the guidance provided herein, using conventional methods.

Non-limiting exemplary dosage forms of the present invention include tablets, capsules, powders, liquids, suspensions, solutions, caplets, lozenges, health bars, soups, cereals, confections, particles, microparticles, granules, beads and animal feeds. These dosage forms are all well-known to persons of ordinary skill in the art. The term “pharmaceutically-acceptable”, as used herein, means that the dosage form must be of sufficiently high purity and suitable for use in contact with cells, tissues, or membranes without undue toxicity, incompatibility, instability, allergic response, and the like.

In a preferred embodiment, the therapeutic composition of the subject invention is administered orally to a biological subject. It is believed that any flavoring or food may be added to alter taste as desired.

Various additives may be incorporated into the present composition. Optional additives of the present composition include, without limitation, starches, sugars, fats, antioxidants, amino acids, proteins, derivatives thereof or combinations thereof.

It is also possible in the pharmaceutical composition of the inventive subject matter for the dosage form to combine various forms of release, which include without limitation, immediate release, extended release, pulse release, variable release, controlled release, timed release, sustained release, delayed release, long acting, and combinations thereof. The ability to obtain immediate release, extended release, pulse release, variable release, controlled release, timed release, sustained release, delayed release, long acting characteristics and combinations thereof is performed using well known procedures and techniques available to the ordinary artisan. Each of these specific techniques or procedures for obtaining the release characteristics is well known to those of ordinary skill in the art. As used herein, a “controlled release form” means any form having at least one component formulated for controlled release. As used herein, “immediate release form” means any form having at least some of its pharmaceutically active components formulated for immediate release. Any biologically-acceptable dosage form, and combinations thereof, are contemplated by the inventive subject matter. Examples of such dosage forms include, without limitation, chewable tablets, quick dissolve tablets, effervescent tablets, reconstitutable powders, elixirs, liquids, solutions, suspensions, emulsions, tablets, multi-layer tablets, bi-layer tablets, capsules, soft gelatin capsules, lard gelatin capsules, caplets, lozenges, chewable lozenges, beads, powders, granules, particles, microparticles, dispersible granules, cachets, douches, suppositories, creams, topicals, inhalants, aerosol inhalants, patches, particle inhalants, implants, depot implants, ingestibles, injectables, infusions, health bars, confections, animal feeds, cereals, cereal coatings, foods, nutritive foods, functional foods and combinations thereof. The preparation of the above dosage forms is well known to persons of ordinary skill in the art.

The following procedures represent, without limitation, acceptable methods of preparing formulations falling within the scope of the inventive subject matter.

Quick dissolve tablets may be prepared, for example, without limitation, by mixing the formulation with agents such as sugars and cellulose derivatives, which promote dissolution or disintegration of the resultant tablet after oral administration, usually within 30 seconds.

Cereal coatings may be prepared, for example, without limitation, by passing the cereal formulation, after it has been formed into pellets, flakes, or other geometric shapes, under a precision spray coating device to deposit a film of active ingredients, plus excipients onto the surface of the formed elements. The units thus treated are then dried to form a cereal coating.

Health bars may be prepared, without limitation, by mixing the formulation plus excipients (e.g., binders, fillers, flavors, colors, etc.) to a plastic mass consistency. The mass is then either extended or molded to form “candy bar” shapes that are then dried or allowed to solidify to form the final product.

Soft gel or soft gelatin capsules may be prepared, for example, without limitation, by dispersing the formulation in an appropriate vehicle (vegetable oils are commonly used) to form a high viscosity mixture. This mixture is then encapsulated with a gelatin based film using technology and machinery known to those in the soft gel industry. The industrial units so formed are then dried to constant weight.

Chewable tablets, for example, without limitation, may be prepared by mixing the formulations with excipients designed to form a relatively soft, flavored, tablet dosage form that is intended to be chewed rather than swallowed. Conventional tablet machinery and procedures, that is both direct compression and granulation, or slugging, before compression, can be utilized. Those individuals involved in pharmaceutical solid dosage form production are well versed in the processes and the machinery used as the chewable dosage form is a very common dosage form in the pharmaceutical industry.

Film coated tablets, for example, without limitation, may be prepared by coating tablets using techniques such as rotating pan coating methods or air suspension methods to deposit a contiguous film layer on a tablet. This procedure is often done to improve the aesthetic appearance of tablets, but may also be done to improve the swallowing of tablets, or to mask an unpleasant odor or taste, or to improve properties of an unsightly uncoated tablet.

Compressed tablets, for example, without limitation, may be prepared by mixing the formulation with excipients intended to add binding qualities to disintegration qualities. The mixture is either directly compressed or granulated, then compressed using methods and machinery quite well known to those in the industry. The resultant compressed tablet dosage units are then packaged according to market need, i.e., unit dose, rolls, bulk bottles, blister packs, etc.

For example, animal feed may be made by methods well known to persons of ordinary skill in the art. Animal feeds may be prepared by mixing the formulation with binding ingredients to form a plastic mass. The mass is then extruded under high pressure to form tubular (or “spaghetti-like”) structures that are cut to pellet size and dried.

The present inventive subject matter contemplates pharmaceutical compositions formulated for administration by any route, including without limitation, oral, buccal, sublingual, rectal, parenteral, topical, inhalational, injectable and transdermal, preferably oral. The physicochemical properties of compositions, their formulations, and the routes of administration are important in absorption. Absorption refers to the process of nutritional composition movement from the site of administration toward the systemic circulation. Orally administered compositions maybe in the form of tablets or capsules primarily for convenience, economy, stability, and patient acceptance. They must disintegrate and dissolve before absorption can occur. Using the present inventive subject matter, with any of the above routes of administration or dosage forms, is performed using well known procedures and techniques available to the ordinary skilled artisan.

The present inventive subject matter contemplates the use of biologically-acceptable carriers which may be prepared from a wide range of materials. Without being limited thereto, such materials include diluents, solvents, binders and adhesives, lubricants, plasticizers, disintegrates, colorants, bulking substances, flavorings, sweeteners and miscellaneous materials, such as buffers and adsorbents in order to prepare a particular medicated composition.

Binders may be selected from a wide range of materials, such as hydroxypropylmethylcellulose, ethylcellulose, or other suitable cellulose derivatives, povidone, acrylic arid methacrylic acid co-polymers, pharmaceutical glaze, gums, milk derivatives, such as whey, starches, and derivatives, as well as other conventional binders well known to persons skilled in the art. Exemplary non-limiting non-toxic solvents are water, ethanol, isopropyl alcohol, methylene chloride or mixtures and combinations thereof. Exemplary non-limiting bulking substances include sugar, lactose, gelatin, starch, and silicon dioxide.

The plasticizers used in the dissolution modifying system are preferably previously dissolved in an organic solvent and added in solution form. Preferred plasticizers may be selected from the group consisting of diethyl phthalate, diethyl sebacate, triethyl citrate, cronotic acid, propylene glycol, butyl phthalate, dibutyl sebacate, caster oil and mixtures thereof, without limitation. As is evident, the plasticizers may be hydrophobic as well as hydrophilic in nature. Water-insoluble hydrophobic substances, such as diethyl phthalate, diethyl sebacate and caster oil are used to delay the release of water soluble materials. In contrast, hydrophilic plasticizers are used when water-insoluble materials are employed which aid in dissolving the encapsulated film, making channels in the surface, which aid in composition release.

The composition of the present inventive subject matter may be administered in a partial, i.e., fractional dose, one or more times during a 24 hour period, a single dose during a 24 hour period of time, a double dose during a 24 hour period of time, or more than a double dose during a 24 hour period of time. Fractional, double or other multiple doses may be taken simultaneously or at different times during the 24 hour period.

The following examples further describe and demonstrate embodiments within the scope of the present invention. The examples are given solely for the purpose of illustration and are not to be construed as limitations of the present invention, as many variations thereof are possible without departing from the spirit and scope of the invention.

A study was conducted to observe the anti-inflammatory and therapeutic properties of emu oil on both auricular (ear) and cutaneous inflammation in male CD-1 mice, induced topically by application of 2% croton oil. In addition, the study was designed to measure the different cytokines levels and to assess whether there is any association between the cytokines levels and the anti-inflammatory effect.

### EXAMPLE 1

To evaluate and measure the levels of different cytokines, a preliminary study was conducted in ten animals. IL-1α, TNF α and IL-6 are considered to be the predominant pro-inflammatory cytokines, in the family of interleukins that play a significant role in inducing any inflammatory response. Inner surface of right auricles (ears) of CD-1 mice were exposed to croton oil followed by application of emu oil at one hour. Animals were sacrificed at different time points: 2, 4, 7 and 9 hours post croton oil application. Two animals served as untreated controls. Exposed and unexposed ears were harvested and weighed at time of sacrifice and frozen in liquid nitrogen and saved at −85 degrees for further analysis. Blood samples were drawn from the heart and smears made for differential counts, to evaluate whether there are any systemic reactions to this inflammatory induction. Serum was harvested from the blood and saved for cytokines levels evaluation.

Detection of mouse interleukins and other cytokines from tissue samples is done in vitro, by Enzyme Linked Immuno Sorbent Assay (ELISA) method. This is an antigen, antibody reaction and it's a quantitative determination of cytokines in mouse serum or tissue extracts (homogenized ear tissue). ELISA kit wells (Biosource, CA) coated with specific antibody are treated with sample sera or tissue extract. After a series of incubations and washing, the reaction is colorimetrically measured. The intensity of colored product is directly proportional to the concentration of the cytokine in the original specimen.

Frozen ears were digested in a tissue homogenizer containing lysis buffer. This process releases the cytokines from the cells. The extract was used to detect the levels of cytokines under consideration, measured by ELISA.

From this preliminary study and the ELISA results, it was concluded that at 5-6 hours post exposure to croton oil, the predominant cytokine responsible for this inflammatory action is IL-1α. The emu oil mediated its' action primarily by suppressing the pro-inflammatory cytokine, IL-1α. The other two cytokines, TNF α and IL-6 do not seem to play a significant role in elucidating this inflammatory action during this period.

Comparison of photographs taken at different time points and comparative measurements of auricular thickness and ear plug weights, strongly support the earlier findings that the anti-inflammatory properties of emu oil was greatest around 6 hours.

Following this preliminary experiment, 34 male CD-1 mice weighing 25 to 28 grams were obtained from Charles River Laboratories (Wilmington, Mass.) and housed in polycarbonate cages and given commercial food and water ad libitum for 15 days before the commencement of the study. On the day of the experiment, body weights ranged from 29 to 32 grams. Animals were cared for, as per the guidelines set forth by the ‘The Guide’ and the Institutional Animal Care and Use Committee.

Mice were randomly assigned to three groups; Control group−untreated (n=11), olive oil control group (n=11) and emu oil treatment group (n=14). Animals were anesthetized with a combination of ketamine (100 mg/ml) and xylazine (20 mg/ml). Dosage: Ketamine/Xylazine 87 mg/kg−13 mg/kg IM. Auricular inflammation was induced by topical application of 2% croton oil to the inner surface of the right auricle. One hour later, the assigned treatment (untreated group, olive oil control group and emu oil treatment group) was carried out to each group. Dorsal skin area was clipped before the application of croton oil to the skin site. Similar treatment was carried out as for the auricles. The left ear and the left cutaneous area remained untreated and served as noninflamed controls.

Auricular thickness was measured prior to the exposure of croton oil using micrometer calipers. Photographs were taken to illustrate the degree of inflammation and the treatment responses seen in each group. Animals were sacrificed at 6 hours post application of emu oil and the thickness of the pinna measured again. Uniform size areas were punched from the treated and control sites (ears and skin) and weighed. At the time of sacrifice, blood samples were drawn from the heart. Blood smears were made for differential cell counts. Serum was harvested from the blood and saved for further cytokine analysis.

Application of 2% croton oil induced observable hyperemic inflammation and swelling in the auricles of CD-1 mice. Photographic evidence clearly illustrates this phenomenon. On the cutaneous site, no detectable hyperemia, swelling or inflammation was seen. There was edematous swelling in the right auricles and the magnitude of swelling was calculated as the weight difference between the right (inflamed) and the left (noninflamed) uniform earplugs. Pre-treatment and post-treatment thickness measurements of auricles show a significant difference in both the control and oil control groups, indicating that there was no anti-inflammatory effect in these groups. In the emu oil group, the thickness measurements (pre and post) were not significantly different. This showed that emu oil was able to reverse the inflammatory process induced by croton oil. It is also well supported by the reversal of hyperemia, compared to the control groups, as seen in the photographs.

Comparison of weights of uniform ear plugs from left and right ears show that in the emu oil group there was very little difference in weight. In the control groups, the difference was very significant (statistical data).

ELISA results from serum indicate that there was no significant difference seen between the control group and the emu oil group. This indicates that the inflammatory action is more local than systemic. We expect to see significant correlation between the anti-inflammatory process and the cytokine levels from the excised ear tissues, indicating that there is suppression of the predominate pro-inflammatory cytokine IL-1α by emu oil application. (Complete results will be available in three weeks time)

As shown by other investigators, application of 5 ul of emu oil at 5-6 hours post croton oil exposure significantly reduced the degree of inflammation in the auricles of CD-1 mice. The inflammatory process reverses itself in 24 hours, if left untreated. The natural host defense actions and mechanisms of the body bring about this reversal process. This is a complex phenomenon involving leukocyte cell migration, attachment of definitive cells to vascular endothelium and the movement of endothelial cells. Neutrophils appear early in the sites where there is acute inflammation and this is in part controlled by cytokine induction. In this study, we looked at the possible relationship and interaction between the different cytokines and the anti-inflammatory process and how emu oil mediates its' anti-inflammatory action.

Final analysis of the cytokine evaluation results will lead us to conclude the hypothesis, that the anti-inflammatory effect of emu oil is directed towards the pro-inflammatory cytokine IL-1α.

On the cutaneous site, there was no inflammation seen to the exposure to croton oil. This may be because the dorsal skin area differs histologically to the auricular site, having additional subcutaneous layers. It is possible that injecting the causative agent subcutaneously may induce a detectable inflammatory response. A pilot study may prove or disprove this theory.

The results of the study are set forth in Tables I-III. Table I shows the ear plug weight differences for the refined emu oil composition as compared to olive oil.

TABLE I
EAR PLUG WEIGHT DIFFERENCES (MG)

| Control | Olive Oil | Emu Oil |
|---|---|---|
| 16.7 | 18.8 | 8.2 |
| 22.9 | 22.6 | 10.6 |
| 15.1 | 16.1 | 5.6 |
| 22.3 | 11.8 | 3.6 |
| 14.8 | 14.5 | 6.6 |
| 14.0 | 17.6 | 1.9 |
| 10.3 | 19.4 | 3.2 |
| 21.2 | 26.2 | 10.4 |
| 27.6 | 26.7 | 6.7 |
| 27.6 | 26.7 | 6.7 |
| 21.3 | 18.9 | 4.7 |
| 26.3 | 24.7 | 3.4 |
| 19.3 ± 5.5^{a} | 19.8 ± 4.8^{a} | 2.0 |
| | | 8.6 |
| | | 3.2 |
| | | 5.6 ± 2.9^{b} |

Values represent Means±Std Dev for 11-14 animals/group

Values with different superscripts are significantly different from each other, at least p<0.001

Table II summaries the results of the emu oil study.

TABLE II
EMU OIL STUDY - SUMMARY OF RESULTS
Thickness difference between Left and Right ears in mm

| | Control © | Olive (O) | Emu oil |
|---|---|---|---|
| | 0.31 | 0.22 | 0.12 |
| | 0.27 | 0.29 | 0.17 |
| | 0.25 | 0.27 | 0.14 |
| | 0.22 | 0.19 | 0.15 |
| | 0.22 | 0.21 | 0.18 |
| | 0.18 | 0.21 | 0.16 |
| | 0.2 | 0.18 | 0.13 |
| | 0.3 | 0.29 | 0.13 |
| | 0.28 | 0.35 | 0.13 |
| | 0.32 | 0.28 | 0.14 |
| | 0.28 | 0.29 | 0.18 |
| | | | 0.17 |
| | | | 0.15 |
| | | | 0.16 |
| AVE | 0.257273 | 0.252727 | 0.150714 |
| SD | 0.04671 | 0.053496 | 0.019793 |
| VAR | 0.002182 | 0.002862 | 0.000392 |

Table III provides detailed data observed and recorded for the ear thickness of each animal during the emu oil study.

TABLE III

| | | EMU OIL STUDY - SUMMARY OF EAR THICKNESS (mm) | | | | | | |
|---|---|---|---|---|---|---|---|---|
| | | | 6 Hours Post Treatment With EMU Oil | | | | | |
| | | | Control | | Olive oil | | | |
| | | Control | Right | Olive oil | Right | Emu oil | Emu oil | Thick. Diff. |
| Date | Animal No. | Left Ear | Ear | Left Ear | Ear | Left Ear | Right Ear | (mm) |
| | | | | | | | | 0.31 |
| 10/12/2000 | 1 | 0.25 | 0.56 | | | | | 0.27 |
| | 2 | 0.25 | 0.52 | | | | | 0.25 |
| | 3 | 0.28 | 0.53 | | | | | 0.22 |
| | 4 | | | 0.3 | 0.52 | | | 0.29 |
| | 5 | | | 0.27 | 0.56 | | | 0.27 |
| | 6 | | | 0.3 | 0.57 | | | 0.12 |
| | 7 | | | | | 0.3 | 0.42 | 0.17 |
| | 8 | | | | | 0.25 | 0.47 | 0.14 |
| | 9 | | | | | 0.28 | 0.42 | 0.22 |
| 10.23.00 | 10 | 0.3 | 0.52 | | | | | 0.22 |
| | 11 | 0.27 | 0.49 | | | | | 0.18 |
| | 12 | 0.31 | 0.49 | | | | | 0.2 |
| | 13 | 0.29 | 0.49 | | | | | 0.19 |
| | 14 | | | 0.32 | 0.51 | | | 0.21 |
| | 15 | | | 0.31 | 0.52 | | | 0.21 |
| | 16 | | | 0.29 | 0.5 | | | 0.18 |
| | 17 | | | 0.31 | 0.49 | | | 0.15 |
| | 18 | | | | | 0.32 | 0.47 | 0.18 |
| | 19 | | | | | 0.29 | 0.46 | 0.16 |
| | 20 | | | | | 0.3 | 0.46 | 0.13 |
| | 21 | | | | | 0.28 | 0.41 | 0.13 |
| 11/17/2000 | 22 | 0.3 | 0.6 | | | | | 0.3 |
| | 23 | 0.27 | 0.55 | | | | | 0.28 |
| | 24 | 0.27 | 0.59 | | | | | 0.32 |
| | 25 | 0.31 | 0.59 | | | | | 0.28 |
| | 26 | | | 0.3 | 0.59 | | | 0.29 |
| | 27 | | | 0.27 | 0.62 | | | 0.35 |
| | 28 | | | 0.29 | 0.57 | | | 0.28 |
| | 29 | | | 0.27 | 0.56 | | | 0.29 |
| | 30 | | | | | 0.33 | 0.46 | 0.13 |
| | 31 | | | | | 0.32 | 0.45 | 0.134 |
| | 32 | | | | | 0.35 | 0.49 | 0.14 |
| | 33 | | | | | 0.29 | 0.47 | 0.18 |
| | 34 | | | | | 0.34 | 0.41 | 0.17 |
| | 35 | | | | | 0.30 | 0.45 | 0.15 |
| | 36 | | | | | 0.31 | 0.46 | 0.16 |

### EXAMPLE 2

### OBJECTIVE

To compare the anti-inflammatory properties

of Emu Oil versus:

Croton Oil

Corn Oil

Olive Oil

Fish Oil

Flaxseed Oil

by measuring:

1. auricular thickness and weight

2. auricular cytokine levels (IL-1a and TNF-a)

### Experimental Design and Methods

The left and right auricle of all anesthetized animals was topically treated with croton oil to induce inflammation. One hour later, after increased auricular thickness was recorded, the right auricle was topically exposed to either croton oil (untreated), corn, olive, fish, flaxseed, chicken fat or emu oil. Six hours later, all animals were euthanized, auricular thickness re-measured and auricular weights determined. Both auricles were then frozen for subsequent analyses of cytokines IL-1a and TNF-a by an ELISA method. Emu oil was observed to have anti-inflammatory properties that are at least equivalent to those reported for omega 3 dietary oils in CD-1 mice.

The present invention has been described in connection with the preferred embodiments. These embodiments, however, are merely for example and the invention is not restricted thereto. Any examples described herein are illustrative of preferred embodiments of the inventive subject matter and are not to be construed as limiting the inventive subject matter thereto. It will be understood by those skilled in the art that other variations and modifications can easily be made within the scope of the invention as defined by the appended claims.

What is claimed is:

1. A composition for reducing and/or preventing inflammation in a patient comprising:
colorless emu oil;
wherein the colorless emu oil reduces and/or prevents inflammation in the patient when administered in an effective amount to said patient.

2. The composition of claim 1, wherein the colorless emu oil has an American Oil Chemist's Society (AOCS) color rating of Cc13b-45 using the Lobibond method.

3. The composition of claim 1, further comprising a pharmaceutically-acceptable carrier.

4. The composition of claim 1, wherein the composition is prepared by a process comprising:
obtaining subcutaneous fat from an emu; and
processing the subcutaneous fat at a low temperature with the absence of oxygen to obtain colorless emu oil.

5. The composition of claim 4, wherein the temperature is about 150 degrees F. to about 200 degrees F.

6. The composition of claim 4, wherein the temperature is about 150 degrees F. to about 180 degrees F.

7. A method for reducing and/or preventing inflammation in a patient comprising:
administering to the patient an effective amount of a composition comprising colorless emu oil.

8. The composition of claim 7, wherein the colorless emu oil has an American Oil Chemist's Society (AOCS) color rating of Cc13b-45 using the Lobibond method.

9. The method of claim 7, wherein the composition is prepared by a process comprising:
obtaining subcutaneous fat from an emu; and
processing the subcutaneous fat at a low temperature with the absence of oxygen to obtain colorless emu oil.

10. The method of claim 9, wherein the temperature is about 150 degrees F. to about 200 degrees F.

11. The method of claim 9, wherein the temperature is about 150 degrees F. to about 180 degrees F.

12. The method of claim 7, wherein the composition additionally comprises a pharmaceutically-acceptable carrier.

13. A method for preparing a composition for reducing and/or preventing inflammation in a patient comprising:
obtaining subcutaneous fat from an emu;
processing the subcutaneous fat at a low temperature with the absence of oxygen to obtain colorless emu oil.

14. The method of claim 13, wherein the temperature is about 150 degrees F. to about 200 degrees F.

15. The method of claim 13, wherein the temperature is about 150 degrees F. to about 180 degrees F.

16. The method of claim 13, wherein the colorless emu oil has an American Oil Chemist's Society (AOCS) color rating of Cc13b-45 using the Lobibond method.
